(19) United States
(12) Patent Application Publication
(10) Pub. No.: US 2010/0113605 A1
(43) Pub. Date: May 6, 2010

(54) METHODS, COMPOSITIONS AND USES THEREOF

(76) Inventor: David John HEAL, Nottingham (GB)

Correspondence Address: FENNEMORE CRAIG, 3003 NORTH CENTRAL AVENUE, SUITE 2600, PHOENIX, AZ, 85012, US

(21) Appl. No.: 12/593,447

(22) PCT Filed: Mar. 28, 2008
(86) PCT No.: PCT/GB2008/001126

§ 371 Date: Nov. 11, 2009

(30) Foreign Application Priority Data
Mar. 29, 2007 (GB) 0706077.5

(51) Int. Cl. A61K 38/27; G01N 33/53; C07K 14/61; A61P 25/00

(52) U.S. Cl. 514/646; 435/7.21; 564/442

(57) ABSTRACT

The invention relates to a method for identifying a candidate compound for treating a disorder or condition associated with dysfunction of monoamine neurotransmission in the central nervous system, the method comprising the following steps: (a) providing a compound to be tested; (b) testing the ability of the compound to bind to the cocaine-binding site of a monoamine reuptake transporter; and (c) testing the ability of the compound to modulate the inward or outward transport of monoamine neurotransmitters via the monoamine reuptake transporter, wherein the test compound is identified as a candidate compound for treating a disorder or condition associated’ with dysfunction of monoamine neurotransmission if it is able to bind to the cocaine-binding site of the monoamine reuptake transporter and modulate its activity. The invention further relates to compounds identified using the method of the invention, and uses, compositions and medicaments thereof.

### METHODS, COMPOSITIONS AND USES THEREOF

The present invention relates to the mechanism of action of monoamine transmission in the central nervous system (CNS) and, in particular, to a method for identifying a candidate compound for treating a disorder or condition associated with dysfunction of monoamine neurotransmission.

Cocaine is a powerful psychostimulant drug of abuse which is extracted from the leaves of the coca shrub (erythroxylon coca). Cocaine can be abused by various means including ingestion, insufflation and smoking and its abuse is a major problem in many developed and developing countries around the world. The burden on society is enormous with the White House Office of National Drug Control Policy estimating that between 1988 and 1995 Americans spent approximately $38 billion on the illicit purchase of this psychostimulant. This figure does not take into account the indirect costs of cocaine abuse, including those related to law enforcement, medical admissions, social support, rehabilitation and lost financial productivity, nor does it encompass the harm to society of the criminal activities linked with the illegal supply and use of cocaine.

The psychostimulant effects of cocaine are believed to derive from its ability to increase the function of the monoamine neurotransmitter, dopamine, in the brain and it is its actions in the limbic regions of the brain that are believed to be responsible for its activating, euphoriant, reinforcing and rewarding properties in man (Di Chiara et al, 1993). Prior to this invention, it was hypothesised that cocaine increased dopaminergic function in the central nervous system by competitively blocking dopamine reuptake transporter (DAT) sites on dopaminergic neurones thereby passively preventing the transport of dopamine out of the synaptic cleft and back into the presynaptic dopaminergic nerve terminal. This mode of action for cocaine on the neuronal monoamine reuptake transporter (originally called Uptake 1) was originally described by Iversen (1973). However, this proposed mechanism does not explain why other dopamine reuptake inhibitors, e.g. bupropion and sibutramine (via its pharmacologically active metabolites) are often more potent as dopamine reuptake inhibitors than cocaine but have no similar psychostimulant euphoriant actions in man (Griffith et al, 1983; Miller & Griffith, 1983; Schuh et al, 2000). Importantly, cocaine addiction and withdrawal have not been successfully treated using such agents (Gorelick et al, 2004; Sofuoglu & Kosten, 2005).

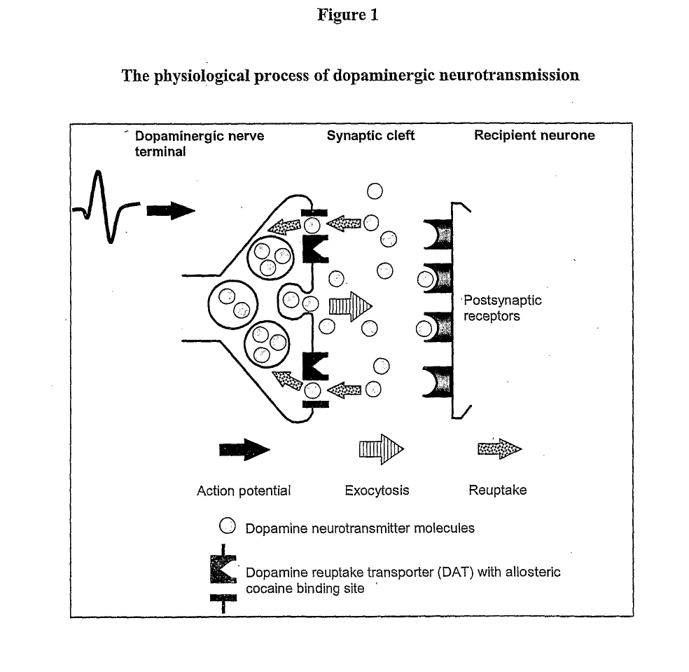

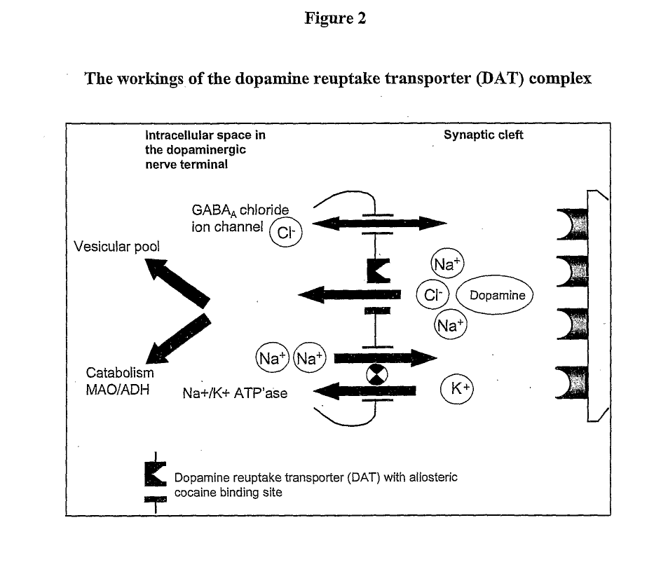

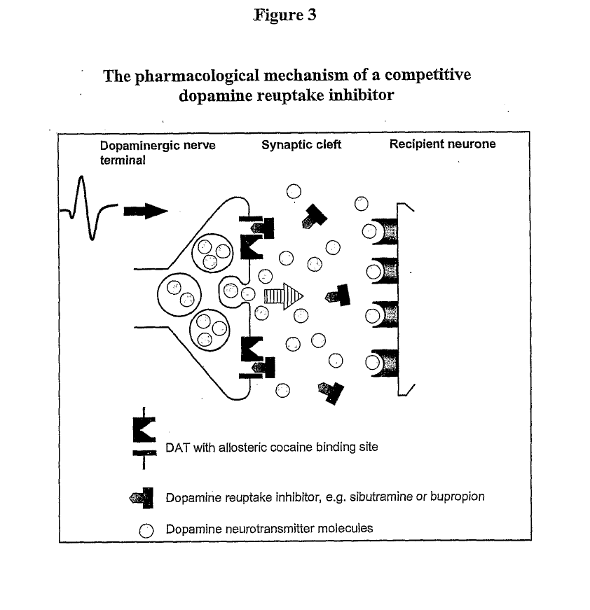

The physiological mechanism for the exocytotic release of dopamine is illustrated in FIG. 1, along with a diagrammatic representation of the role of DAT in the modulation of dopaminergic neurotransmission. An increased rate of dopaminergic neuronal firing leads to the exocytotic release of dopamine from dopamine-containing nerve terminals into the synaptic cleft. This chemical messenger then transmits its signal to a recipient (postsynaptic) neurone via receptors located on it. The primary physiological mechanism for terminating dopaminergic signalling is the removal of the neurotransmitter from the synaptic cleft by a process of active reuptake via the sodium/chloride ion channel DAT (FIG. 2). As shown in FIG. 3, competitive reuptake inhibitors, e.g. sibutramine (via its active metabolites) and bupropion, block this process and it results in a gradual, moderate and prolonged increase in dopamine concentrations in the synaptic cleft thereby gradually and moderately enhancing dopaminergic neurotransmission. The competitive reuptake inhibitor has no direct effect on dopaminergic neurotransmission, it merely potentiates and prolongs the actions of exocytotically released dopamine.

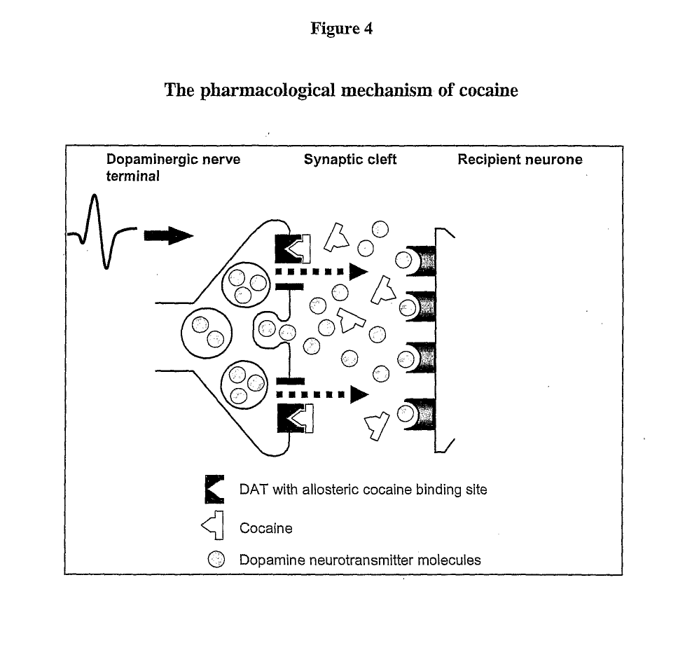

In contrast, as shown in FIG. 4, cocaine and pharmacologically-related compounds, e.g. methylphenidate which evokes cocaine-like psychostimulant and euphoriant effects in man (Rush & Baker, 2001), act at a separate site on DAT, i.e. the “cocaine binding site” (Edvardsen & Dahl, 1994), to enhance dopaminergic neurotransmission. Although cocaine has long been known to bind to this site, it is widely believed that it merely acted as a competitive DAT inhibitor, (cf sibutramine's metabolites and bupropion) passively to block the clearance of dopamine from the synapse via these transporters.

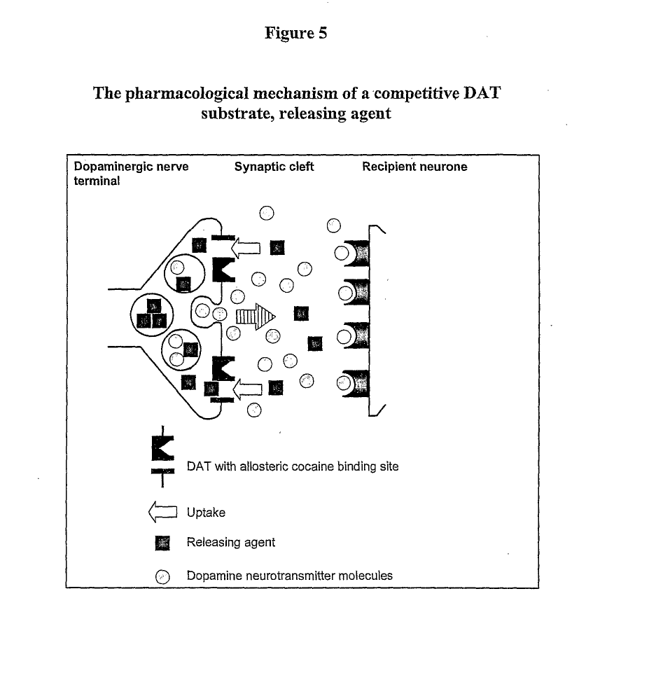

The competitive DAT substrate-releasing agents, e.g. d-amphetamine, methamphetamine, methylamphetamine, methylenedioxyamphetamine (MDA) and methylenedioxymethamphetamine (MDMA), are all powerful stimulators of dopaminergic neurotransmission in the central nervous system (Table 1). These molecules are similar to dopamine in their size and 3-dimensional structure. As shown in FIG. 5, these releasing agents are competitive substrates for the DAT complex, which pumps them into dopamine-containing nerve terminals. Once inside the presynaptic terminal, they displace dopamine from the “releasable” (newly synthesised) and vesicular storage pools and forcibly expel this neurotransmitter into the synaptic cleft by displacement. Because the releasing agents are substrates for the DAT complex, they also delay the clearance of dopamine from the synaptic cleft by competing with it for transport into the presynaptic nerve terminal. The competitive DAT substrate releasing agents produce their pharmacological actions predominantly from within the nerve terminal, and in addition, their effects on dopaminergic neurotransmission are independent of neuronal firing.

A summary of the similarities and the key differences between the pharmacological characteristics of these various different DAT ligands is shown in Table 2.

Against this background, the inventor has surprisingly discovered that cocaine is not a conventional competitive dopamine reuptake inhibitor, but instead acts as an inverse agonist at the “cocaine binding site” on the DAT complex, This much more powerful dynamic pharmacological mechanism, which reverses the transport of dopamine so that it is pumped out of the dopaminergic nerve terminal, explains why cocaine and related drugs have serious psychostimulant abuse liability.

The term “inverse agonist” was first coined by in the 1980's by Polc et al (1982) to describe the actions of a novel class of benzodiazepine ligands, e.g. FG-7142. The benzodiazepine agonists are known to bind to a modulatory site on the γ-aminobutyric acid (GABA) A-type chloride ion channel receptor where they increase Cl⁻ ion flux into nerve cells and by this mechanism are anticonvulsant and anxiolytic. Their actions can be blocked by antagonists, e.g. Ro 15-1788, which themselves have no effect on Cl⁻ ion fluxes and are, therefore, described as being pharmacologically “silent”. The actions of ligands like FG-7142, on the other hand, were observed to be the opposite of those of the benzodiazepine agonists; thus, they decreased Cl⁻ ion flux into neurones, they were proconvulsant and profoundly anxiogenic. Since their actions were the inverse of the benzodiazepine agonists, they were logically described as “inverse agonists”. There are now known to be inverse agonists for a number of other receptor systems, including G-protein coupled receptors. However, this invention is the first description of an “inverse agonist” for a transporter; in this case, the sodium/chloride ion DAT that is present on dopamine-containing neurones in the brain where it plays a pivotal role in regulating dopaminergic neurotransmission.

The evidence presented herein demonstrates that the “cocaine binding site” on DAT is an allosteric, modulatory site on this transporter, and here, cocaine and related compounds act as inverse agonists to produce transport of dopamine molecules out of the nerve terminal into the synaptic cleft. By this dynamic mechanism, cocaine profoundly increases dopaminergic neurotransmission in the brain. This action of cocaine and related compounds is exerted outside of the dopaminergic nerve terminal and is dependent on intact dopaminergic nerve firing.

This is a surprising feature of the pharmacological mechanism of action for cocaine and related “cocaine binding site” ligands and indicates that cocaine produces its psychostimulant and euphoriant effects by acting as an inverse agonist at the dopamine reuptake transporter (DAT).

Although most research has focussed on the action of cocaine on DAT, the monoamine family of transmembrane transporters also includes serotonin (SERT) and norepinephrine (NET) transporters (Amara and Arriza, 1993). These three molecules are all Na⁺/Cl⁻-dependent monoamine reuptake sites which share a high amino acid homology (Blakely et al, 1991; Giros et al, 1991, 1992; Pacholczyk at al, 1991; Ramamoorthy at al, 1993). Cocaine is able to bind with high affinity to DAT, NET and SERT (Hyttel, 1982; Richelson & Pfenning, 1984; see Table 3).

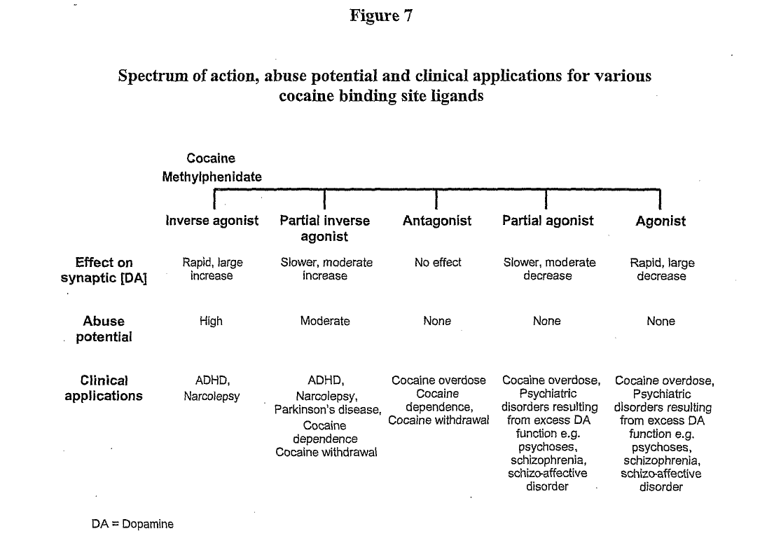

This invention and the experimental methods employed in its realisation have an application as a method for the screening and pharmacological characterisation of other cocaine binding site ligands, i.e. inverse agonists, partial inverse agonists, agonists, partial agonists and antagonists, as novel drugs for the treatment of cocaine overdose, cocaine craving, cocaine addiction and the physical and psychological syndrome produced on withdrawal from cocaine abuse (FIG. 7). Furthermore, the invention has therapeutic application to the development of novel cocaine binding site ligands, i.e. inverse agonists, partial inverse agonists, agonists, partial agonists and antagonists, as drugs for the treatment of clinical disorders associated with psychostimulant abuse and also to psychiatric and neurological diseases and conditions resulting from deficiencies or excesses of dopaminergic function in the brain (FIG. 7).

The present invention is also applicable to the discovery and development of novel drugs for the treatment of overdose, craving, addiction and the withdrawal syndromes produced by other psychostimulant drugs of abuse including, but not limited to, amphetamine, methamphetamine, MDA and MDMA and their isomers and congeners. An additional benefit of this invention is it provides a method for pharmacologically manipulating dopaminergic neurotransmission in the brain in both directions, i.e. upwards and downwards, whilst still retaining physiological rates of dopaminergic neuronal firing. This modality can be applied to the development of novel drugs to treat psychiatric and neurological disorders that result either from deficits or excesses of dopaminergic neurotransmission. Examples of conditions resulting from deficits in dopaminergic neurotransmission include, but are not limited to, attention deficit hyperactivity disorder (ADHD) and related CNS disorders of cognition, impulsiveness, attention and aggression, narcolepsy and Parkinson's disease. Examples of conditions resulting from an excess of dopaminergic neurotransmission include, but are not limited to, schizophrenia, schizo-affective disorder and related psychoses.

Accordingly, a first aspect of the invention provides a method for identifying a candidate compound for treating a disorder or condition associated with dysfunction of monoamine neurotransmission in the central nervous system, the method comprising the following steps:
a) providing a compound to be tested;
b) testing the ability of the compound to bind to the cocaine-binding site of a monoamine reuptake transporter; and
c) testing the ability of the compound to modulate the inward or outward transport of monoamine neurotransmitters via the monoamine reuptake transporter;
   wherein the test compound is identified as a candidate compound for treating a disorder or condition associated with dysfunction of monoamine neurotransmission if it is able to bind to the cocaine-binding site of the monoamine reuptake transporter and modulate its activity.

Thus, step (c) of the method of the first aspect of the invention comprises testing the activity of the monoamine reuptake transporter, in particular by testing the inward or outward transport of monoamine neurotransmitters.

In one embodiment, step (c) comprises testing the ability of the compound to modulate the activity of the cocaine binding site of the monoamine reuptake transporter. Thus, step (c) may comprise testing the ability of the compound to modulate the action of cocaine on the monoamine reuptake transporter.

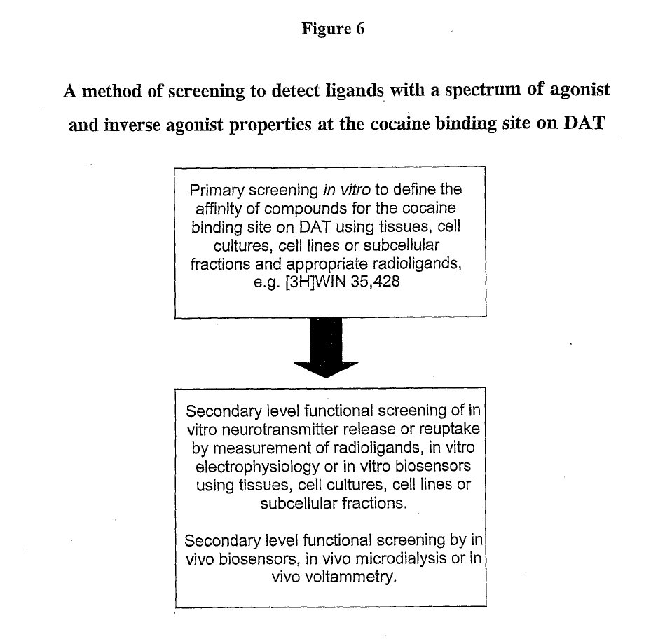

The invention therefore provides a screening strategy for the identification and pharmacological differentiation of novel drugs that are inverse agonists, partial inverse agonists, antagonists, partial agonists and full agonists of the cocaine binding site on the DAT complex (FIG. 6, Tables 4 and 5).

As detailed in Table 2, the ligands for the cocaine binding site on the DAT complex have unique pharmacological characteristics which differentiate them from both the competitive DAT reuptake inhibitors and competitive DAT substrate releasing agents. In the light of the unexpected discovery that cocaine is an inverse agonist at the DAT complex, it is, therefore, evident that as a result of its dynamic effect on this sodium/chloride ion transporter that compounds can display a range of pharmacological actions from those of inverse agonists (that reverse the direction of the DAT transporter) through silent antagonists (that will have no pharmacological effect on the functioning of the DAT transporter) through to full agonists (that will markedly increase the rate of clearance of dopamine from the synaptic cleft). However, as demonstrated in the current invention, the actions of drugs at the cocaine binding site are dependent on the physiological rates of neuronal firing of the dopaminergic neurones. Consequently, conventional functional screening techniques, e.g. the inhibition of [³H]dopamine uptake into synaptosomes, are not applicable to the detection and pharmacological characterisation of molecules with these dynamic characteristics. This is because in synaptosomal and cell-line preparations, intact neuroanatomy and the physiological dopaminergic nerve firing, which are essential for the dynamic actions of cocaine binding site ligands on the DAT complex, are absent. As described in the accompanying Examples, the in vivo techniques of intracerebral microdialysis and voltammetry and electrically-stimulated neuronal, fast-cyclic voltammetry in conscious or anaesthetised rats led to the unexpected discovery that the cocaine binding site is an allosteric, modulatory subunit on the DAT complex and when cocaine binds to this site it acts as an inverse agonist to reverse the normal direction of dopamine transport. Because of the dynamic nature of this interaction and its reliance on intact dopaminergic neuronal firing, such experimental techniques need to be applied to the discovery and pharmacological characterisation of all cocaine binding site ligands. FIG. 6 shows how conventional screening assays, e.g. radioligand receptor binding, are currently employed to define the affinity of a ligand for the cocaine binding site in tissues or cell-lines stably expressing the DAT complex. However, such techniques do not define the function of the compound in question, e.g. inverse agonist, partial inverse agonist, antagonist, partial agonist or agonist. As described in Tables 4 and 5, this objective can be achieved by employing the in vivo techniques described above and the relevant outputs to define the pharmacological characteristics of novel cocaine binding site ligands are defined therein.

Preferably, the invention provides a method wherein the monoamine reuptake transporter is selected from the group consisting of reuptake transporters of dopamine, noradrenaline and/or serotonin (5-HT).

Thus, in one embodiment, the monoamine reuptake transporter is a dopamine reuptake transporter.

For example, the invention may provide a method wherein the disorder or condition is associated with a deficit of dopamine neurotransmission in the central nervous system; in particular, the disorder or condition is selected from the group comprising or consisting of Parkinson's disease, narcolepsy, attention deficit hyperactivity disorder (ADHD), borderline personality disorder, intermittent explosive disorder, antisocial personality disorder, substance abuse, kleptomania and pyromania.

Alternatively, the disorder or condition may be associated with an excess of dopamine neurotransmission in the central nervous system, such as a disorder or condition selected from the group comprising or consisting of schizophrenia, schizo-affective disorder, schizophreniform disorder, substance abuse-induced psychotic disorder, delusional disorder, mania and shared psychotic disorder.

In a further embodiment, the monoamine reuptake transporter is a noradrenaline reuptake transporter.

For example, the invention may provide a method wherein the disorder or condition is associated with a deficit of noradrenaline neurotransmission in the central nervous system, such as a disorder or condition is selected from the group comprising or consisting of disorders of impulsiveness, attention and aggression, for example attention deficit hyperactivity disorder (ADHD), borderline personality disorder, intermittent explosive disorder, antisocial personality disorder, substance abuse, kleptomania, pyromania and depression., substance abuse, kleptomania, pyromania and depression.

Alternatively, the disorder or condition may be associated with an excess of noradrenaline neurotransmission in the central nervous system, such as a disorder or condition is selected from the group comprising or consisting of panic attacks, post-traumatic stress disorder, anxiety, phobias and obsessive-compulsive disorder.

In a further embodiment, the monoamine reuptake transporter is a serotonin reuptake transporter.

For example, the invention may provide a method wherein the disorder or condition is associated with a deficit of serotonin neurotransmission in the central nervous system, such as a disorder or condition is selected from the group comprising or consisting of disorders of impulsiveness, attention and/or aggression, for example borderline personality disorder, intermittent explosive disorder, antisocial personality disorder, substance abuse, kleptomania, pyromania, eating disorders (binge eating, bulimia, anorexia), anxiety, phobias, obsessive-compulsive disorder and depression.

Alternatively, the invention provides a method wherein the disorder or condition is associated with an excess of serotonin neurotransmission in the central nervous system, for example migraine.

In a preferred embodiment of the invention, step (b) is performed by in vitro receptor binding using appropriate radioligands, e.g. [³H]WIN35,428 (e.g. Aloyo et al, 1995; Chen et al, 1996; Katz et al, 2000), and step (c) may be performed by in vitro neurotransmitter release or reuptake using tissue slices, cells or synaptosomes (e.g. de Langen & Mulder, 1980; Pristupa et al, 1994; Pifl et al, 1995; Heal et al, 1996; Sershen et al, 1996; Rowley et al, 2000), in vitro electrophysiology (e.g. Jones et al, 1996; Cragg et al, 2000), in vivo microdialysis (e.g. Rowley et al, 2000), in vivo voltammetry (e.g. Wu et al, 2001), in vitro biosensors or implanted biosensors in vivo (e.g. Crespi et al 1990; Allen 1997)

It will, be appreciated by persons skilled in the art that step (c) of the methods of the invention may comprise testing the ability of the compound to modulate passively and/or actively the activity of the monoamine reuptake transporter.

In one embodiment, the invention provides a method in which step (c) comprises testing the ability of the compound to modulate passively the activity of the monoamine reuptake transporter (and/or the cocaine-binding site thereof).

For example, step (c) may comprise testing the ability of the compound to act as an antagonist of the monoamine reuptake transporter (and/or the cocaine-binding site thereof).

In a further embodiment, the invention provides a method in which step (c) comprises testing the ability of the compound to modulate actively the activity of the monoamine reuptake transporter (and/or the cocaine-binding site thereof).

For example, step (c) may comprise testing the ability of the compound to act as an inverse agonist (either full or partial) of the monoamine reuptake transporter. Such inverse agonism at the cocaine binding site of a monoamine reuptake blocker may be characterised by the following properties:
(I) the compound is capable of inducing and/or increasing neuronal cell-firing-dependent release of the monoamine; and
(ii) the compound has no effect on the re-uptake rate of the monoamine.

Cell-firing-dependent release of the monoamine may be determined by in vivo microdialysis measurements of monoamine efflux. For example, cell firing can be inhibited by perfusion of the microdialysis probe with tetrodotoxin or EGTA.

The reuptake rate of the monoamine may be determined by measurement of labelled monoamine transport into synaptosomes.

Alternatively, or in addition, step (c) may comprise testing the ability of the compound to act as an agonist (either full or partial) of the monoamine reuptake transporter.

Alternatively, or in addition, step (c) may comprise testing the ability of the compound to antagonise the effect of agonists or inverse agonists of the monoamine reuptake transporter (and/or the cocaine-binding site thereof).

It will be appreciated by persons skilled in the art that step (c) may comprise or consist of testing the ability of the compound to modulate the activity of the monoamine reuptake transporter in vitro and/or in vivo.

In a preferred embodiment, step (c) comprises or consists of testing the ability of the compound to act at the cocaine binding site on the monoamine reuptake transporter. For example, step (c) may comprise or consist of testing the ability of the compound to act as an agonist (full or partial) at the cocaine binding site on the dopamine reuptake transporter.

The effects of drugs on the extraneuronal concentrations of dopamine (a surrogate for their effects on the synaptic concentrations of this neurotransmitter) can be evaluated using various in vitro and in vivo techniques, including for example, the in vitro release of [³H]dopamine from preloaded brain slices measured by superfusion, measurement of extraneuronal dopamine concentrations in the brains of freely-moving rats by intracerebral microdialysis coupled with high performance liquid chromatography (HPLC) plus electrochemical detection or in vivo intracerebral fast-cyclic voltammetry. Data obtained from this combination of techniques reveal that cocaine and related compounds do not function either as competitive DAT reuptake inhibitors, cf sibutramine (via its active metabolites) and bupropion, or competitive substrate releasing agents, cf amphetamine and methamphetamine; rather they have a unique pharmacological mode of action that is consistent with inverse agonism at an allosteric, modulatory site on the DAT complex and it is the cocaine binding site. Thus in summary, cocaine differs from the competitive DAT reuptake inhibitors because at high concentration it releases [³H]dopamine from preloaded brain slices, and as measured by intracerebral microdialysis in vivo, it evokes very large increases in extraneuronal dopamine concentrations that are very rapid in onset and of relatively short duration. Cocaine is pharmacologically different from competitive substrate-releasing agents because although both classes of compound evoke very large increases in extraneuronal dopamine concentrations in vivo as shown by intracerebral microdialysis experiments, the actions of cocaine are dependent on dopaminergic neuronal firing, whilst those of the competitive substrate releasing agents are not. Finally, in vivo voltammetry experiments demonstrate that cocaine increases the rate of dopamine release evoked by electrical stimulation as well as the maximum quantity of neurotransmitter released, but this drug does not delay the rate of dopamine clearance from the synaptic cleft. The inventive steps in this application have been to deduce from these two observations that the cocaine binding site is not merely a location on the DAT complex where cocaine binds passively to block the uptake of dopamine; rather it is an allosteric, modulatory site that controls the rate and direction of transport of dopamine in and out of the dopaminergic neurone. Moreover, it has been shown here that cocaine acts as an inverse agonist at the cocaine binding site on DAT to potentiate firing-evoked dopamine efflux. If by attaching to the cocaine binding site, cocaine functioned as a reuptake inhibitor, it would slow the rate of clearance of this neurotransmitter, which the data show it does not.

Typically, step (c) comprises testing the ability of the compound to modulate the activity of the monoamine reuptake transporter using one or more of the following techniques including, but not limited to:
(A) In vitro measurement of spontaneous monoamine release from tissue slices, cells (or a subcellular fraction thereof) containing monoamine reuptake transporter sites by superfusion (e.g. de Langen & Mulder, 1980; Pristupa et al, 1994; Pifl et al, 1995; Heal et al, 1996);
(B) In vitro measurement of monoamine reuptake from tissue slices, cells (or a subcellular fraction thereof) containing monoamine reuptake transporter sites by superfusion (e.g. de Langen & Mulder, 1980; Pristupa et al, 1994; Pifl et at, 1995; Heal et al, 1996);
(C) In vitro measurement of electrically-evoked release of monoamine from tissue slices, cells (or a subcellular fraction thereof) containing monoamine reuptake transporter sites by superfusion (e.g. Sershen at al, 1996);
(D) In vitro measurement of spontaneous and/or electrically-evoked monoamine efflux from tissue slices, cells (or a subcellular fraction thereof) containing monoamine reuptake transporter sites by electrophysiological techniques (e.g. Jones et al, 1996; Cragg at al, 2000);
(E) In vitro and/or in vivo measurement of spontaneous and/or electrically evoked monoamine efflux using one or more biosensors (e.g. Crespi at al 1990; Allen 1997);
(F) In vivo measurement of cell-firing dependent and cell-firing independent monoamine efflux by microdialysis in animals (e.g. Rowley et al, 2000);
(G) In vivo measurement of spontaneous and/or electrically-evoked monoamine efflux in animals by voltammetric techniques (e.g. Wu et al, 2001).

In a preferred embodiment, (A), (B) and (C) comprise the in vitro measurement of release of a labelled monoamine.

In a further preferred embodiment, the biosensors of (E) are coated with for example, enzymes, antibodies and/or neurotransmitter receptors.

It will be appreciated by persons skilled in the art that the method of the invention may comprise one or more of the following technique options/combinations:
A Alone, B alone, C alone, D alone, E alone, F alone, G alone, A+B, A+C, A+D, A+B+C, A+B+C+D, A+B+D, B+C, B+D, C+B, C+D, A+C+E (in vitro), A+C+E (in vivo), A+C+F, A+C+G, A+B+C+E (in vitro), A+B+C+E (in vivo), A+B+C+F, A+B+C+G, A+D+E (in vitro), A+D+E (in vivo), A+D+F, A+D+G A+B+D+E (in vitro), A+B+D+E (in vivo), A+B+D+F, A+B+D+G

It will be further appreciated that any tissue, cell or subcellular fraction containing monoamine reuptake transporter sites could be used in the method of the invention. For example, the cells containing monoamine reuptake transporter sites may be in or derived from tissue slices, such as brain slices (e.g. the basal ganglia area)

Preferably, the invention provides a method wherein the tissue slices are from the brain, for example from dopaminergic regions of the brain

The cells containing monoamine reuptake transporter sites may also be maintained in culture. Thus, the cells may be selected from the group consisting of primary cells and immortalised cells (i.e. cell lines), which may be genetically modified to express a monoamine reuptake transporter. Suitable methods, for genetically modifying cells are described in Sambrook & Russell, 2001, Molecular Cloning: A Laboratory Manual, Cold Spring Harbor Press.

In one embodiment, the cells containing monoamine reuptake transporter sites are blood cells.

Alternatively, the cells containing monoamine reuptake transporter sites are in or derived from renal blood vessels.

Alternatively, monoamine release or reuptake may be measured in a subcellular fraction, such as synaptosomes.

The cells containing monoamine reuptake transporter sites may be derived from any suitable source, for example a human or a non-human (e.g. a rodent such as a mouse or a rat).

In the methods of the invention comprising in vivo assessment of the function of monoamine reuptake transporters, such measurements are preferably performed in the brain. For example, the in vivo measurements are performed in regions of the brain rich in cells which contain monoamine reuptake transporters. Typically, monoamine reuptake transporter function may be measured in vivo in dopaminergic regions of the brain (e.g. the basal ganglia).

Preferably, the in vivo measurements are performed in a human or a non-human species (e.g. a rodent such as a mouse or a rat).

Techniques suitable for detecting monoamine release and reuptake are known in the art. Preferably, radioligand counting, autoradiography, HPLC (combined with, for example, fluorescence detection, electrical detection, mass spectrometry detection) immunoassays, fluorescence detection, electrical detection, mass spectrometry detection and enzyme assays could be used in the method of the invention.

In one embodiment, step (c) of the method of the invention comprises testing the ability of the test compound at different doses to modulate the activity of the monoamine reuptake transporter. For example, in vitro measurement of release of monoamine from tissue by superfusion may be performed using a high dose of the test compound (e.g. 10⁻⁵ M) and a low dose of the test compound (e.g. 10⁻⁷ M).

It is preferred that the method of the invention further comprises counter-screening the test compounds for adverse or undesirable properties, for example toxicity and/or abuse potential.

Advantageously, the method further comprises step (d) of formulating a compound identified as a candidate compound for treating a disorder or condition associated with dysfunction of monoamine neurotransmission in the central nervous system into a pharmaceutical composition.

In a further aspect, the invention provides a compound identified by a method according to the method of the invention.

For example, the compound may be a full or partial inverse agonist of the cocaine binding site of a monoamine reuptake transporter. Alternatively, the compound may be a full or partial agonist of the cocaine binding site of a monoamine reuptake transporter. In a further embodiment, the compound is an antagonist of ligands (such as cocaine) which act as inverse agonists of the cocaine binding site of a monoamine reuptake transporter.

In a further aspect, the invention provides a pharmaceutical composition comprising a compound according to the invention and a pharmaceutically-acceptable carrier or excipient.

The compounds, medicaments and pharmaceutical compositions of the present invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

An alternative method of delivery of the compounds, medicaments and pharmaceutical compositions of the invention is the ReGel injectable system that is thermo-sensitive. Below body temperature, ReGel is an injectable liquid while at body temperature it immediately forms a gel reservoir that slowly erodes and dissolves into known, safe, biodegradable polymers. The active substance, is delivered over time as the biopolymers dissolve.

Preferably, the medicament and/or pharmaceutical composition of the present invention is a unit dosage containing a daily dose or unit, daily sub-dose or an appropriate fraction thereof, of the active ingredient.

The compounds, medicaments and pharmaceutical compositions of the invention will normally be administered orally or by any parenteral route, in the form of a pharmaceutical composition comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds, medicaments and pharmaceutical compositions of the invention can be administered alone but will generally be administered in admixture with a suitable pharmaceutical excipient, diluent or carrier selected with regard to the intended route of administration and standard pharmaceutical practice.

For example, the compounds, medicaments and pharmaceutical compositions of the invention can be administered orally, buccally or sublingually in the form of tablets, capsules, ovules, elixirs, solutions or suspensions, which may contain flavouring or colouring agents, for immediate-, delayed- or controlled-release applications. The compounds, medicaments and pharmaceutical compositions of the invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds, medicaments and pharmaceutical compositions of the invention can also be administered parenterally, for example, intravenously, intra-arterially, intraperitoneally, intra-thecally, intratracheally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Medicaments and pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The medicaments and compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds, medicaments and pharmaceutical compositions of the invention may be administered in single or divided doses.

Thus, for example, the tablets or capsules of the compound of the invention may contain sufficient active agent for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds, medicaments and pharmaceutical compositions of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichloro-tetrafluoroethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active agent, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an agent of the invention and a suitable powder base such as lactose or starch.

Aerosol or dry powder formulations are preferably arranged so that each metered dose or “puff” contains the compound of the invention for delivery to the patient. It will be appreciated that the overall daily dose with an aerosol will vary from patient to patient, and may be administered in a single dose or, more usually, in divided doses throughout the day.

Alternatively, the compounds, medicaments and pharmaceutical compositions of the invention can be administered in the form of a suppository or pessary, or they may be applied topically in the form of a lotion, solution, cream, ointment or dusting powder. The compounds, medicaments and pharmaceutical compositions of the invention may also be transdermally administered, for example, by the use of a skin patch.

For application topically to the skin, the compounds, medicaments and pharmaceutical compositions of the invention can be formulated as a suitable ointment containing the active agent suspended or dissolved in, for example, a mixture with one or more of the following: mineral oil, liquid petrolatum, white petrolatum, propylene glycol, polyoxyethylene polyoxypropylene agent, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or parenteral administration of the compounds, medicaments and pharmaceutical compositions of the invention agents of the invention is the preferred route, being the most convenient.

For veterinary use, the compounds, medicaments and, pharmaceutical compositions of the invention is administered as a suitably acceptable formulation in accordance with normal veterinary practice and the veterinary surgeon will determine the dosing regimen and route of administration which will be most appropriate for a particular animal.

Conveniently, the formulation is a pharmaceutical formulation.

Advantageously, the formulation is a veterinary formulation.

The present invention further provides a compound as described herein for use in medicine. Thus, the invention provides the use of a compound according to the invention in the manufacture of a medicament for treating a disorder or condition associated with dysfunction of monoamine neurotransmission in the central nervous system.

For example, the disorder or condition may be associated with dysfunction of reuptake transporters of dopamine, noradrenaline and/or serotonin (5-HT).

Thus, in one embodiment, the monoamine reuptake transporter is a dopamine reuptake transporter.

For example, the invention may provide a treatment method wherein the disorder or condition is associated with a deficit of dopamine neurotransmission in the central nervous system; in particular, the disorder or condition is selected from the group comprising or consisting of Parkinson's disease, narcolepsy, attention deficit hyperactivity disorder (ADHD), borderline personality disorder, intermittent explosive disorder, antisocial personality disorder, substance abuse, kleptomania and pyromania.

Alternatively, the disorder or condition may be associated with an excess of dopamine neurotransmission in the central nervous system, such as a disorder or condition selected from the group comprising or consisting of schizophrenia, schizo-affective disorder, schizophreniform disorder, substance abuse-induced psychotic disorder, delusional disorder, mania and shared psychotic disorder.

In a further embodiment, the monoamine reuptake transporter is a noradrenaline reuptake transporter.

For example, the invention may provide a treatment method wherein the disorder or condition is associated with a deficit of noradrenaline neurotransmission in the central nervous system, such as a disorder or condition is selected from the group comprising or consisting of disorders of impulsiveness, attention and aggression, for example attention deficit hyperactivity disorder (ADHD), borderline personality disorder, intermittent explosive disorder, antisocial personality disorder, substance abuse, kleptomania, pyromania and depression., substance abuse, kleptomania, pyromania and depression.

Alternatively, the disorder or condition may be associated with an excess of noradrenaline neurotransmission in the central nervous system, such as a disorder or condition is selected from the group including, but not limited to panic attacks, post-traumatic stress disorder, anxiety, phobias and obsessive-compulsive disorder.

In a further embodiment, the monoamine reuptake transporter is a serotonin reuptake transporter.

For example, the invention may provide a treatment method wherein the disorder or condition is associated with a deficit of a deficit of serotonin neurotransmission in the central nervous system, such as a disorder or condition is selected from the group comprising or consisting of disorders of impulsiveness, attention and/or aggression, for example borderline personality disorder, intermittent explosive disorder, antisocial personality disorder, substance abuse, kleptomania, pyromania, eating disorders (binge eating, bulimia, anorexia), anxiety, phobias, obsessive-compulsive disorder and depression.

Alternatively, the invention provides a method wherein the disorder or condition is associated with an excess of serotonin neurotransmission in the central nervous system, for example migraine.

The listing or discussion of a prior-published document in this specification should not necessarily be taken as an acknowledgement that the document is part of the state of the art or is common general knowledge.

Preferred, non-limiting examples which embody certain aspects of the invention will now be described, with reference to the following tables and figures:

Table 1: Effects of various DAT ligands on [³H]dopamine release from rat striatal slices.

Table 2: Classification of various types of DAT ligand.

Table 3: A comparison of the relative potency of cocaine as a reuptake inhibitor of radiolabelled dopamine, noradrenaline and 5-HT

Table 4: Characteristics of various DAT cocaine binding site ligands using an intracerebral microdialysis or voltammetry functional screen.

Table 5: Characteristics of various DAT cocaine binding site ligands in an electrically-stimulated, fast cyclic voltammetry screen in vivo.

FIG. 1: The physiological process of dopaminergic neurotransmission. An action potential (nerve impulse) travelling along the axon of a dopaminergic neurone depolarises it leading to the quantal release of dopamine from its storage vesicles into the synaptic cleft by a process called exocytosis. The chemical messenger, dopamine, diffuses across the synaptic cleft on to the recipient neurone where dopamine transmits its signal by activating post-synaptic receptors. Dopaminergic signalling is predominantly terminated by removing this neurotransmitter from the synaptic cleft by an ionic gradient-driven active transport via the dopamine reuptake transporter (DAT) sites on presynaptic dopaminergic nerve terminals.

FIG. 2: The workings of the dopamine reuptake transporter (DAT) complex. Dopamine is pumped back into the presynaptic terminal by the ionic gradient-linked active transporter, DAT. The reuptake transporter binds dopamine, 2 sodium and 1 chloride ions and translocates them into the presynaptic terminal. The ionic gradient powering the system is maintained by sodium/potassium ATPase and modulatory chloride ion channels, e.g. GABA_{A} receptors.

FIG. 3: The pharmacological mechanism of a competitive dopamine reuptake inhibitor. Competitive dopamine reuptake inhibitors, e.g. sibutramine or bupropion, bind competitively to the dopamine reuptake transporter thereby preventing the clearance of dopamine from the synaptic cleft by DAT. This passive effect leads to a gradual and moderate increase in the concentration of dopamine in the synaptic cleft.

As shown in the figure, competitive dopamine reuptake inhibitors potentiate dopaminergic neurotransmission from outside of the nerve terminal and their actions are dependent on the rate of neuronal firing. Reuptake inhibitors have no direct pharmacological actions; they merely potentiate and prolong the effect of physiologically released dopamine.

FIG. 4: The pharmacological mechanism of cocaine. Unlike the classical reuptake inhibitors, e.g. sibutramine and bupropion, which competitively block the transport of dopamine into the nerve terminal via dopamine, cocaine binds to an allosteric site on the DAT complex (the cocaine binding site). Cocaine as an inverse agonist reverses the function of the transporter from a mechanism to transport dopamine into the nerve terminal to one where it actively transports dopamine out of it. The result is a rapid and very large increase in synaptic dopamine concentrations, and thus, dopaminergic neurotransmission; this mechanism accounts for cocaine's profoundly psychostimulant profile.

As shown on the figure, cocaine potentiates dopaminergic neurotransmission by binding to DAT sites which are outside of the nerve terminal and its action is dependent on neuronal firing.

FIG. 5: The pharmacological mechanism of a competitive DAT substrate, releasing agent. Competitive DAT substrate releasing agents e.g. d-amphetamine and methamphetamine, are small molecules that mimic the endogenous monoamine transmitter, dopamine. They are actively transported into the presynaptic nerve terminals via the DAT complex. Once inside the neurone, they displace dopamine from its storage sites and forcibly release it into the synaptic cleft by a process called “reverse transport” or “retro transport”. Dopamine releasing agents also delay the clearance of this monoamine from the synaptic cleft by competing with it for transport into the dopaminergic nerve terminal.

As shown in the figure, competitive DAT substrate releasing agents potentiate dopaminergic neurotransmission from inside the nerve terminal and their actions are independent of neuronal firing.

FIG. 6: A method of screening to detect ligands with a spectrum of agonist and inverse agonist properties at the cocaine binding site on DAT.

FIG. 7: Spectrum of action, abuse potential and clinical applications for various cocaine binding site ligands.

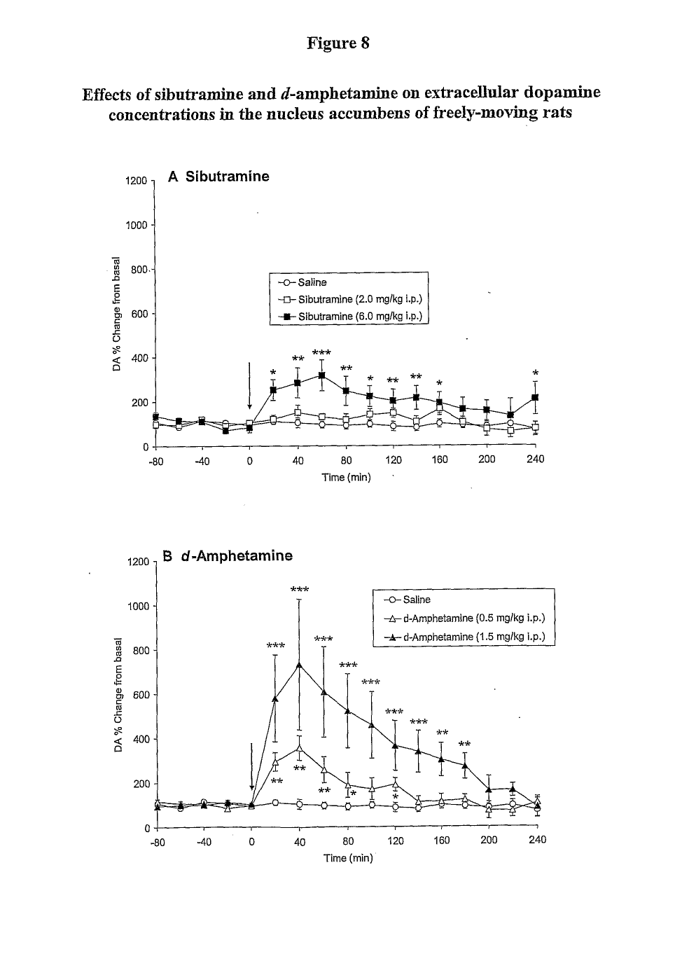

FIG. 8: Effects of sibutramine and d-amphetamine on extracellular dopamine concentrations in the nucleus accumbens of freely-moving rats. Each data point represents mean±S.E.M. (n=8-11); sibutramine, d-amphetamine or saline administration is indicated by the vertical arrow. *P<0.05; **P<0.01; ***P<0.001 significantly different from saline-treated group according to ANCOVA with post hoc t-test for multiple comparisons. Data taken from Rowley et al (2000).

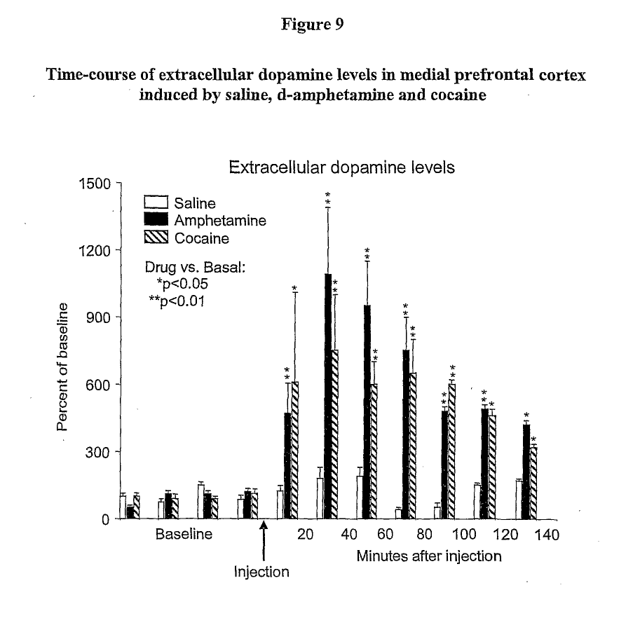

FIG. 9: Time-course of extracellular dopamine levels in medial prefrontal cortex induced by saline, d-amphetamine and cocaine. Time-course of extracellular dopamine levels in medial prefrontal cortex induced by saline, d-amphetamine (1.25 mg/kg in females, 1.56 mg/kg in males) and cocaine (20 mg/kg). Samples were collected over 20 min intervals. Data are expressed as a percent (±SEM) of baseline values. Data are taken from Maisonneuve et al (1990).

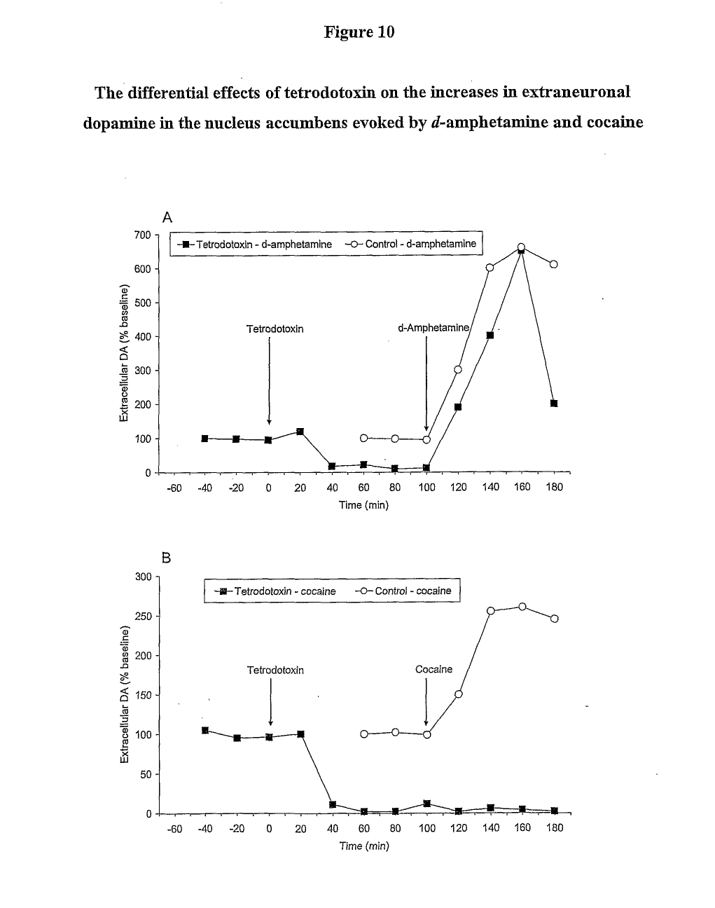

FIG. 10: The differential effects of tetrodotoxin on the increases in extraneuronal dopamine in the nucleus accumbens evoked by d-amphetamine and cocaine. The Ringer solution used to dialyse the probes was changed to one containing tetrodotoxin (1×10⁻⁶M: n=4) at the point indicated by the first arrow. All of the animals were given injections of d-amphetamine (0.5 mg/kg s.c.) or cocaine (15 mg/kg i.p.) at the point indicated by the second arrow (n=7 for the animals tested using the standard Ringer). The results are expressed as means of the pretreatment values measured in the three samples collected prior to switching the Ringer solution (filled squares) or prior to the injection of d-amphetamine or cocaine (open circles). Basal levels of dopamine in the dialysate (uncorrected for recovery through the probe), were 0.062±0.014 pmol/20 μl. Data are taken from Westerink at al (1987).

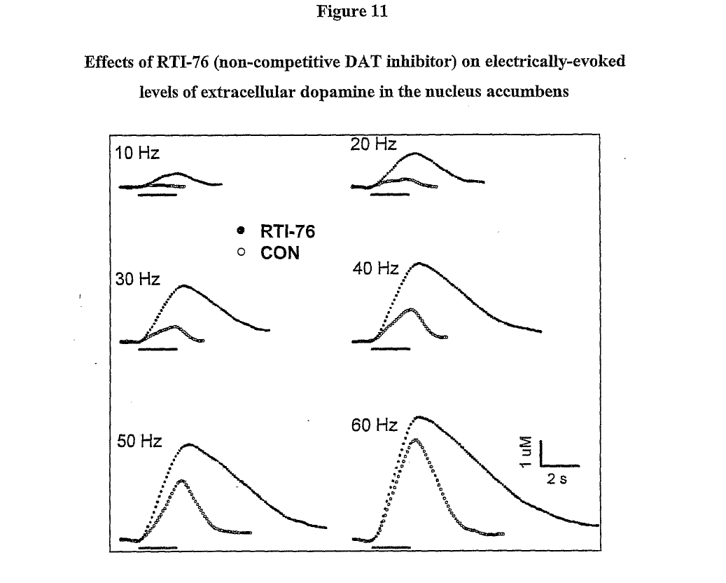

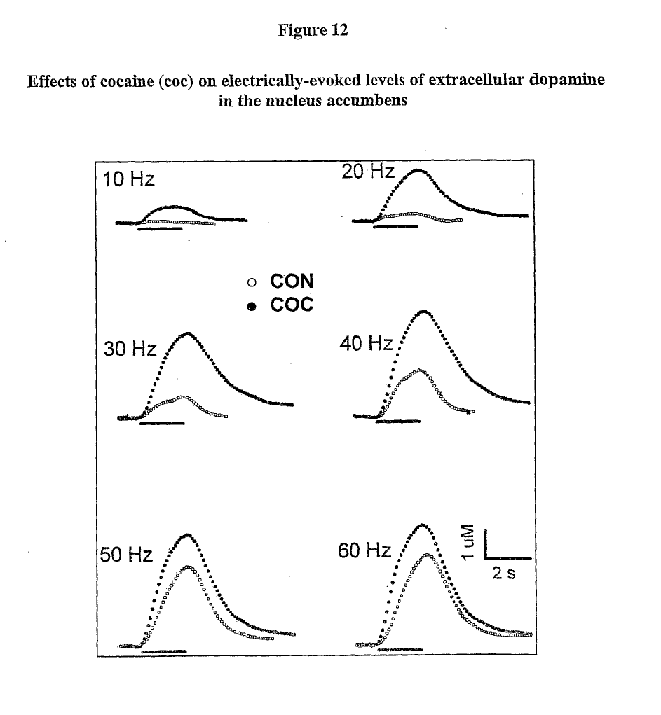

FIG. 11: Effects of RTI-76 on electrically-evoked levels of extracellular dopamine in the nucleus accumbens. Two sets of evoked signals, recorded in different animals, are shown. The first set of traces describes changes in the dopamine signal monitored 1 day after intracerebroventricular injection of RTI-76 (100 nmol; solid circles). The second set (CON; open circles) was recorded in a naive rat. For comparison, time and concentration scales and presentation of data are identical in this figure and FIG. 12. Data are taken from Wu et al (2001).

FIG. 12: Effects of RTI-76 on electrically-evoked levels of extracellular dopamine in the nucleus accumbens. Two sets of evoked signals, recorded in different animals, are shown. The first set of traces describes changes in the dopamine signal monitored 1 day after intracerebroventricular injection of RTI-76 (100 nmol; solid circles). The second set (CON; open circles) was recorded in a naive rat. For comparison, time and concentration scales and presentation of data are identical in this figure and FIG. 12. Data are taken from Wu et al (2001).

### EXAMPLES

### Measurement by Superfusion of the Effects of Various DAT Ligands on [³H]Dopamine Release from Rat Striatal Slices In Vitro

The method employed is that described in detail by Heal et al (1992). Briefly adult male CD rats (180-300 g) were killed, the striata were removed rapidly and slices were prepared by chopping the tissue in 2 directions at 90° using a McIlwain tissue chopper. The slices were then incubated for 20 min at 37° C. in 2 ml Krebs-Henseleit buffer (188 mM NaCl, 25 mM NaHCO₃, 11 mM D-glucose, 4.7 mM KCl, 1.2 mM MgSO₄, 1.2 mM KH₂PO₄, 1.3 mM CaCl₂ gassed with 95% O₂—5% CO₂) pH 7.4 containing 0.13 mM pargyline and 60 nM [³H]dopamine (45 Ci/mmol). Aliquots of 5 mg of slices were transferred to individual chambers of the superfusion apparatus and perfused with Krebs-Henseleit buffer that had been prewarmed to 37° C. The flow-rate was 1 ml/min and after an initial 30 min perfusion had been performed to remove the extraneuronal [³H]dopamine from the slices, fractions were collected at 2 min intervals. The overflow of [³H]dopamine was collected for 8 min (aliquots 1-4) to define the baseline level of [³H]dopamine overflow followed by solutions of test compounds (10⁻⁷-10⁻⁵M) or Krebs-Henseleit buffer alone. Finally, all slices were perfused for a further 10 min. The radioactivity in the fractions and the slices was determined by liquid scintillation counting. The accumulated release of [³H]dopamine present in aliquot numbers 5-13 was then calculated as a fraction of the total radioactivity initially present in the slices.

The test compounds that were evaluated in the experiments described by Heal at al (1992 and 1996) included sibutramine, its active metabolites, i.e. BTS 54 354 (N-{1-[1-(4-chlorophenyl)cyclobutyl]-3-methylbutyl}-N-methylamine) and BTS 54 505 (1-[1-(4-chlorophenyl)cyclobutyl]-3-methylbutylamine), bupropion, cocaine dl-threo-methylphenidate, d-amphetamine and methamphetamine.

### Measurement by Intracerebral Microdialysis of the Effects of Various DAT Ligands on Extraneuronal Dopamine Concentrations in the Nucleus Accumbens of Freely-Moving Rats

Microdialysis experiments were performed as described in detail by Rowley et al (2000). Briefly, male Sprague-Dawley rats (weight range 250-350 g) were anaesthetised with isoflurane in an O₂/N₂O mixture (1 L/min each). A concentric microdialysis probe (300 μM outer diameter) with a 2 mm Hospal membrane tip was stereotaxically implanted into the nucleus accumbens (coordinates: A: +2.2 mm; L: −1.5 mm relative to bregma; −8.0 mm relative to the skull surface according to the stereotaxic atlas of Paxinos and Watson, 1986) and secured to the skull using stainless steel screws and dental cement. After surgery, the rats were individually housed with the microdialysis probe connected via a liquid swivel and counterbalanced arm to ensure the animals' free movement. The probes were continuously perfused with artificial CSF (150 mM Na⁺, 3.0 mM K⁺, 0.8 mM Mg²⁺, 1.4 mM Ca²⁺, 1.0 P²⁺, 155 mM Cl⁺) at a flow-rate of 1.2 μl/min. During the experiment, samples were collected at 20 min intervals into 0.1 M perchloric acid and were then stored at 4° C. until the concentration of dopamine was determined by high performance liquid chromatography (HPLC) coupled with electrochemical detection.

After collection of 4×20 min basal samples, drugs were administered by intraperitoneal injection and 20 min dialysate samples were collected over the following 4 h. The drugs investigated included sibutramine hydrochloride monohydrate (2.0 or 6.0 mg/kg ip) and d-amphetamine sulphate (0.5 or 1.5 mg/kg ip)

Other dialysis data included in this application are as described by Maisonneuve et al (1990), who compared the effects of intraperitoneal injections of d-amphetamine sulphate (1.25 mg/kg in females and 1.56 mg/kg in males) versus cocaine hydrochloride (20 mg/kg ip) on extraneuronal concentrations of dopamine in the medial prefrontal cortex of chloral hydrate- or pentobarbital-anaesthetised Long-Evans rats. Also, as described by Westerink et al (1987), who compared the effects of the sodium channel blocker tetrodotoxin which prevents neuronal firing, on the increased concentrations of dopamine in the nucleus accumbens evoked by cocaine and d-amphetamine.

### Measurement by Fast Cyclic Voltammetry of the Effect of Cocaine on Electrically-Evoked Exocytosis of Dopamine in Rat Nucleus Accumbens in Vivo

These experiments are described in detail in the publication of Wu et al (2001). Briefly, adult male Sprague-Dawley rats (weight range 250-400 g) were anaesthetised by injecting urethane (1.5 g/k ip). Under stereotaxic control, two working electrodes were implanted, with one in the core region of the caudate-putamen and the other in the core region of the nucleus accumbens. A stimulating microelectrode was implanted in the ipsilateral forebrain bundle. The location of dopaminergic neurones was determined by lowering the stimulating electrode until a strong signal was recorded in both the caudate-putamen and nucleus accumbens during a 60 Hz, 2 sec, 300 μA stimulation. The reference electrode was implanted contralaterally in the superficial cortex. After optimising the set-up, the electrode positions were not changed during the entire period of recording. The stimulating electrode was a twisted bipolar electrode with a 0.2 mm tips separated by 1.0 mm. The electrode was insulated along its entire length with the exception of these tips. Electrical stimulation was computer generated and synchronised to the voltammetry measurements. Constant current, biphasic square wave pulses were applied (300-400 μA and 2 msec each phase). The duration of the stimulus trains was 2 sec with frequencies between 10 and 60 Hz randomly applied. Extraneuronal dopamine was quantified using a cylinder carbon fibre microelectrode (exposed tip: radius=2.5 μm, length=50-100 μm). Electrochemistry was computer-controlled and used a potentiostat with provision for 2 working electrodes. A triangular wave (−400 to 1000 mV; 300 v/sec scan rate) was applied every 100 msec. The bias potential between scans was −400 mV. All potentials were referenced to a silver/silver chloride electrode. The extraneuronal concentration of dopamine was obtained from the current at the peak oxidation potential for dopamine (typically 500-700 mV) in successive voltammograms and converted to concentration on the basis of the in vitro calibration of each working electrode after the in vivo experiment. Background-subtracted cyclic voltammograms were obtained by subtracting the voltammograms collected during stimulation from those collected during baseline recording. The analogue output of the potentiostat was digitalised and stored on computer.

Cyclic scanning voltammetry was performed beginning 20 min after injection of cocaine (40 mg/kg ip) and 1 or 2 day after intracerebroventricular injection of RTI-76 (3β-(ρ-chlorophenyl)tropan-2α-carboxylic acid ρ-isothio-cyanatophenylmethyl ester hydrochloride; 100 nmol in 10 μl).

### Results

The Effects of Various DAT Ligands on [³H]Dopamine Release from Rat Striatal Slices In Vitro by Superfusion

The effects of various DAT ligands on the release of [³H]dopamine from superfused rat striatal slices are reported in Table 4. The competitive DAT reuptake inhibitors, sibutramine, its 2 active metabolites (BTS 54 354 and BTS 54 505) and bupropion, did not release [³H]dopamine from striatal slices at either low (10⁻⁷M) or high (10⁻⁵M) concentration. In contrast, the competitive DAT substrate releasing agents, i.e. d-amphetamine and methamphetamine, dose-dependently released [³H]dopamine from striatal slices and this effect was statistically significant at both low and high concentration. The profiles of cocaine and methylphenidate were different from both other types of DAT ligand with no stimulation of [³H]dopamine overflow from striatal slices at low (10⁻⁷M) concentration, but a statistically significant increase at high (10⁻⁵M) concentration (Heal et al, 1992).

### Comparison of the Effects of Sibutramine, D-Amphetamine and Cocaine on Extraneuronal Dopamine Concentrations in the Rat Brain as Determined by Intracerebroventricular Microdialysis

As shown in FIG. 8A, when rats were given injections of the competitive DAT inhibitor, sibutramine, it caused a slow and gradual rise in the extraneuronal concentration of dopamine in the nucleus accumbens of freely-moving rats. At the lower dose of this drug (2.0 mg/kg ip), the increases failed to reach statistical significance. However, at the higher dose (6.0 mg/kg ip), a maximum increase of 231±87% was observed at 60 min (P<0.001). These effects on extraneuronal dopamine in the nucleus accumbens were very different from those observed with pharmacologically equivalent doses of the competitive DAT substrate releasing agent, d-amphetamine (0.5 and 1.5 mg/kg ip). Thus, as shown in FIG. 8B, the effects of d-amphetamine on extraneuronal dopamine concentrations in the nucleus accumbens were more rapid in onset reaching a maximum for both doses of this drug at 40 min. Moreover, the peak increases of 242±89% with the 0.5 mg/kg and 603±319% with the 1.5 mg/kg doses were significantly greater than those observed with sibutramine at these early time-points, i.e. 0-40 min and 40-80 min (P<0.05). These results shown in FIGS. 8A and 8B clearly illustrate the differences between the dynamics of the actions on neuronal dopamine that exist between competitive DAT inhibitors and competitive DAT substrate releasing agents (Rowley et al, 2000).

The data shown in FIG. 9, taken from the study published by Maissoneuve et al (1990), show that in terms of rapidity of onset and magnitude of effect, there is relatively little to differentiate between the actions of the competitive DAT substrate releasing agent, d-amphetamine, and the DAT cocaine binding site ligand, cocaine. However, also using microdialysis techniques, Westerink et al (1987) have shown that the effects of competitive DAT substrate releasing agents like d-amphetamine on extraneuronal dopamine concentrations are not dependent on dopaminergic neuronal firing because they are not altered by inclusion in the dialysate of the sodium channel blocker, tetrodotoxin, which switches-off neuronal firing (FIG. 10A). In contrast, the potentiating effects of cocaine on synaptic dopamine concentrations are prevented by inclusion of tetrodotoxin in the dialysate demonstrating that its pharmacological is totally dependent on intact dopaminergic neuronal firing (Westerink et al, 1987; FIG. 10B).

### Comparison of the Effects of Cocaine and RTI-76 on Extraneuronal Dopamine Concentrations in the Rat Nucleus Accumbens as Determined by Fast Cyclic Voltammetry

RTI-76 is a non-competitive DAT inhibitor, and as clearly shown in FIG. 11, when administered to rats (100 nmol icv), this compound increases the extracellular concentration of dopamine in the nucleus accumbens in a frequency-dependent manner. Consistent with reuptake inhibition as its mechanism of action, the clearance of dopamine by DAT (indicated by the slope of the voltammogram after the peak) is clearly delayed (Wu et al, 2001).

In contrast, as shown in FIG. 12, the action of cocaine on extraneuronal dopamine concentrations in the nucleus accumbens were very different with large increases in the peak concentrations of this neurotransmitter being observed at all stimulus frequencies; however the rate of clearance from the synaptic cleft, i.e. the slopes of the voltammogram after the peaks, were generally parallel to those of the controls indicating that cocaine is increasing synaptic dopamine concentrations via a firing-dependent augmentation of release. These data also show that cocaine was not acting as a reuptake inhibitor to delay the clearance of dopamine from the synapse (Wu et al, 2001).

### Discussion

When these data are viewed overall, it is evident that the pharmacological actions of cocaine and other DAT cocaine binding site ligands, e.g. methylphenidate, on dopaminergic function in the brain differ from those of the other pharmacological classes of DAT ligands, ie competitive DAT reuptake inhibitors exemplified by sibutramine's metabolites and bupropion, and competitive DAT substrate releasing agents exemplified by d-amphetamine, MDA and MDMA. Thus, the cocaine binding site inverse agonists, cocaine and methylphenidate, can be differentiated from the competitive DAT reuptake inhibitors by their ability in vitro moderately to increase [³H]dopamine overflow from preloaded rat striatal slices at high concentration. Slices were employed because they retain neuroanatomical architecture and it is likely that some physiological neuronal firing occurs within this tissue preparation to permit the firing-dependent effects of these cocaine binding site ligands on DAT to occur. This dependence on intact dopaminergic neuronal firing for cocaine-evoked increases in extraneuronal dopamine concentrations has been demonstrated by the in vivo microdialysis experiments of Westerink et al (1987; FIG. 10B). Similarly, cocaine and methylphenidate can also be differentiated from competitive DAT substrate releasing agents, e.g. d-amphetamine and methamphetamine, in vitro because the latter cause profound release of [³H]dopamine from preloaded rat striatal slices and this effect is manifest at very low drug concentrations. This observation is consistent with the dopamine release mechanism of these releasing agents being independent of neuronal firing. This hypothesis has been confirmed in vivo by intracerebral microdialysis experiments which have shown that blocking dopaminergic neuronal firing by infusion of sodium channel blocker, tetrodotoxin, via the dialysis probe does not prevent these releasing agents from enhancing synaptic dopamine concentrations (Westerink et al, 1987; FIG. 10A).

In summary, these in vitro and in vivo findings demonstrate that cocaine and methylphenidate have a pharmacological mechanism that is clearly different from that of other DAT ligands. However, none of the authors of these publications, viz Westerink at (1987), Maissoneuve et at (1990), Heal et al (1992, 1996) or Rowley at (2000) made the inventive step of deducing that cocaine and related compounds are inverse agonists of the DAT complex; merely, that they acted either as reuptake inhibitors or possibly as releasing agents with an unspecified mechanism of action.

Several of the findings from the in vivo cyclic voltammetry study by Wu and colleagues (2001) have been included in support of this patent application, but once again, these authors concluded that cocaine was acting only as a conventional competitive DAT reuptake inhibitor as exemplified by sibutramine's metabolites and bupropion. In their report, Wu et al (2001) noted that cocaine had no effect on the rate of clearance of dopamine from the synaptic cleft. [Ex: “One interesting result of this phenomenon was that the extracellular clearance rate of DA evoked by electrical stimulation, shown previously to reflect Vₘₐₓ for dopamine uptake primarily (see Eq. 3) (Wightman at al, 1988), was not markedly affected by cocaine. In fact, the portion of the evoked response describing the clearance of extracellular DA was essentially parallel to the predrug response at high concentrations (>1 μM).” Wu at al, 2001, p. 6340, R/H column, para 3, lines 4-6 and p 6341, LH column, para 1, lines 1-4]. These authors also noted other unusual findings: first, that the potentiating action of cocaine on synaptic dopamine concentrations was inversely correlated with the number of DAT sites. [Ex: “The relationship is best depicted in FIG. 8 that shows an inverse correlation between inhibitor-induced increases in extracellular DA levels and [DA]ₚ a rate constant for dopamine release, and Vₘₐₓ, which is proportional to the number of DA uptake sites.” Wu et al, 2001, p 6345, L/H column, para 2, lines 4-8] and second, there was an unexplained correlation between the actions of cocaine on dopamine release and its competitive inhibitory action mediated via blockade of dopamine reuptake transporter sites. [Ex: “The observed correlation between DA release and uptake inhibitor effects is surprising because a direct action of the drugs on release is not indicated by the kinetic analysis (FIG. 5). “Wu et al, 2001, p 6345, L/H column, para 3, lines 1-3]. They are contradictory findings which Wu at al (2001) could not reconcile with their postulate that cocaine was a competitive dopamine reuptake transporter inhibitor. The inventive steps made in this patent have been to deduce that the cocaine binding site on the DAT is a modulatory, allosteric subunit of the complex that controls both the rate and direction of dopamine transport. Second, it is to deduce that as the normal direction of transport of dopamine by DAT is into the presynaptic terminal cocaine it is functioning as an inverse agonist at this site to reverse the direction of dopamine transport, i.e. out of the nerve terminal, not into it. Therefore, if cocaine reverses the direction of dopamine transport via the DAT complex by a mechanism that is dependent on intact neuronal firing, the greater the number of DAT sites, the greater the effect of cocaine to augment the exocytotic release of dopamine. Because cocaine is acting as an inverse agonist to augment dopamine release rather than as a competitive dopamine reuptake inhibitor, it has no effect on the clearance of dopamine from the synaptic cleft. At high firing rates, which in turn are associated with higher levels of exocytotic dopamine release, the amount of neurotransmitter stored in the releasable presynaptic pools of within the presynaptic terminal that is available for transport into the synaptic cleft through cocaine's inverse agonism at DAT sites will be markedly reduced. Thus, when the exocytotic release of dopamine is relatively small at low firing rates, it will be markedly augmented by cocaine's inverse agonist action, whereas at higher firing rates, cocaine's contribution to synaptic dopamine concentrations relative to exocytotic release is smaller; this is precisely what is shown by the data of Wu et al (2001) in FIG. 12.

In summary, these experimental findings taken from a wide range of sources have led to the discovery of an unexpected, novel mechanism of action for cocaine and related cocaine binding site ligands. This invention and the experimental methods employed in its discovery have applications for the screening and pharmacological characterisation of other ligands that bind to the cocaine binding site on the DAT complex. Finally, the invention has therapeutic applications to the discovery and development of drugs for the treatment of clinical conditions associated with psychostimulant abuse and also to psychiatric and neurological disorders resulting from deficiencies or excesses of dopaminergic function in the brain.

### REFERENCES

Allen T G (1997). The ‘sniffer-patch’ technique for detection of neurotransmitter release. Trends Neurosci, 20; 192-197.
Aloyo V J, Ruffin J S, Pazdalski P S, Kirifides A L, Harvey J A (1995). [³H]WIN 35,428 binding in the caudate nucleus of the rabbit: evidence for a single site on the dopamine transporter. J Pharmac Exp Ther, 273: 435-444.
Amara S G, Arriza J L (1993). Neurotransmitter transporters: three distinct gene families. Curr Opin Neurobiol 3: 337-344.
Blakely R D, Berson H E, Fremeau Jr R T, Caron M G, Peek M M, Prince H K, Bradley C C (1991). Cloning and expression of a functional serotonin transporter from rat brain. Nature 354: 66-70.
Chen N-H, Xu C, Coffey L L, Reith M E A (1996). [³H]WIN 35,428 [2β-Carbomethoxy-3β-(4-fluorophenyl)tropane] binding to rat brain membranes. Biochem Pharmacol, 51: 563-566.
Cragg S J, Hille C J, Greenfield S A (2000). Dopamine release and uptake dynamics within nonhuman primate striatum in vitro. J Neurosci, 20: 8209-8217.
Crespi F (1990) In vivo voltammetry with micro-biosensors for analysis of neurotransmitter release and metabolism. J Neurosci Methods, 34: 53-65
de Langen C D J, Mulder A H (1980). Effects of psychotropic drugs on the distribution of ³H-dopamine into compartments of rat striatal synaptosomes and on subsequent depolarization-induced ³H-dopamine release. Naunyn Schmiedeberg's Arch Pharmacol, 311: 169-177.
Di Chiara G, Acquas E, Tanda G, Cadoni C (1993). Drugs of abuse: biochemical surrogates of specific aspects of natural reward? Biochem Soc Symp, 59: 65-81.
Edvardsen O, Dahl S G (1994). A putative model of the dopamine transporter. Mol Brain Res, 27: 265-274.
Giros B, el Mestikawy S, Bertrand L, Caron M G (1991). Cloning and functional characterization of a cocaine-sensitive dopamine transporter. FEBS Lett 295: 149-154.
Giros B, el Mestikawy S, Godinot N, Zheng K, Han H, Yang-Feng T, Caron M G (1992). Cloning, pharmacological characterization, and chromosome assignment of the human dopamine transporter. Mol Pharmacol 42: 383-390.
Gorelick D A, Gardner E L, Xi Z X (2004). Agents in development for the management of cocaine abuse. Drugs, 64: 1547-1573.
Griffith J D, Carranza J, Griffith C, Miller L (1983). Bupropion: clinical assay for amphetamine-like abuse potential. J Clin Psychiatry, 44: 206-208.
Heal D J, Frankland A T, Gosden J, Hutchins L J, Prow M R, Luscombe G P, Buckett W R (1992). A comparison of the effects of sibutramine hydrochloride, bupropion and methamphetamine on dopaminergic function: Evidence that dopamine is not a pharmacological target for sibutramine. Psychopharmacol, 107: 303-309.
Heal D J, Prow M R, Hearson M, Buckett W R (1996). Efflux of [³H]dopamine from superfused rat striatal slices: Predictive value for detecting stimulant drugs of abuse. Br J Pharmacol, 117: 325 P.
Hyttel J (1982). Citalopram—Pharmacological profile of a specific serotonin uptake inhibitor with antidepressant activity. Prog Neuro-Psychopharmacol Biol Psychiatr 6: 277-295.
Iversen L L (1973). Catecholamine uptake processes. Br Med Bull, 29: 130-135.
Jones S R, Lee T H, Wightman R M, Ellinwood E H (1996). Effects of intermittent and continuous cocaine administration on dopamine release and uptake regulation in the striatum: in vitro voltammetric assessment. Psychopharmacol, 126: 331-338.
Katz J L, lzenwasser S, Terry P (2000). Relationships among dopamine transporter affinities and cocaine-like discriminative-stimulus effects. Psychopharmacol, 148, 90-98.
Maisonneuve I M, Keller R W, Glick S D (1990). Similar effects of d-amphetamine and cocaine on extracellular dopamine levels in medial prefrontal cortex of rats. Brain Res, 535: 221-226.
Miller L, Griffith J D (1983). A Comparison of bupropion, dextroamphetamine, and placebo in mixed-substance abusers. Psychopharmacol, 80: 199-205.
Pacholczyk T, Blakely R D, Amara S G (1991). Expression cloning of a cocaine- and antidepressant-sensitive human noradrenaline transporter. Nature 350: 350-354
Paxinos G, Watson C (1986). The Rat Brain in Stereotaxic Coordinates. Academic Press, London.
Pifl C, Drobny H, Reither H, Hornykiewicz O, Singer E A (1995). Mechanism of the dopamine-releasing actions of amphetamine and cocaine: plasmalemmal dopamine transporter versus vesicular monoamine transporter. Mol Pharmacol, 47: 368-373.
Polc P, Bonetti E P, Schaffner R, Haefely W (1982). A three-state model of the benzodiazepine receptor explains the interactions between the benzodiazepine antagonist Ro 15-1788, benzodiazepine tranquilizers, beta-carbolines, and phenobarbitone. Naunyn Schmiedebergs Arch Pharmacol, 321: 260-4.
Pristupa Z B, Wilson J M, Hoffman B J, Kish S J, Niznik H B (1994). Pharmacological heterogeneity of the cloned and native human dopamine transporter: disassociation of [³H]WIN 35,428 and [³H]GBR 12,935 binding. Mol Pharmacol, 45: 125-135.
Ramamoorthy S, Bauman A L, Moore K R, Han H, Yang-Feng T, Chang A S, Ganaphthy V, Blakely R D (1993). Antidepressant- and cocaine-sensitive human serotonin transporter: molecular cloning, expression, and chromosomal localization. Proc Natl Aced Sci USA 90: 2542-2546.
Richelson E, Pfenning M (1984). Blockade by antidepressants and related compounds of biogenic amine uptake into rat brain synaptosomes: most antidepressants selectively block norepinephrine uptake. Eur J Pharmacol 104: 227-286.
Rowley H L, Butler S A, Prow M R, Dykes S G, Aspley S, Kilpatrick I C, Heal D J (2000). Comparison of the effects of sibutramine and other weight-modifying drugs on extracellular dopamine in the nucleus accumbens of freely moving rats. Synapse, 38: 167-176.
Rush C R, Baker R W (2001). Behavioural pharmacological similarities between methylphenidate and cocaine in cocaine abusers. Exp Clin Psychopharmacol, 9: 59-73.
Sambrook & Russell (2001) Molecular Cloning: A Laboratory Manual, Cold Spring Harbor Press.
Schuh L M, Schuster C R, Hopper J A, Mendel C M (2000). Abuse liability assessment of sibutramine, a novel weight control agent. Psychopharmacol, 147: 339-346.
Sershen H, Hashim A, Lajtha A (1996). Effect of ibogaine on cocaine-induced efflux of [3H] dopamine and [3H] serotonin from mouse striatum. Pharmacol Biochem Behav, 53: 863-869.
Sofuoglu M, Kosten T R (2005). Novel approaches to the treatment of cocaine addiction. CNS Drugs, 19: 13-25.
Westerink B H, Tuntler J, Damsma G, Rollema H, de Vries J B (1987). The use of tetrodotoxin for the characterization of drug-enhanced dopamine release in conscious rats studied by brain dialysis. Naunyn Schmiedebergs Arch Pharmacol, 336: 502-7.
Wu Q, Reith M E A, Kuhar M J, Carroll F I and Garris P A (2001). Preferential increases in nucleus accumbens dopamine after systemic cocaine administration are caused by unique characteristics of dopamine neurotransmission. J Neurosci, 21: 6338-6347.

TABLE 1
Effects of various DAT ligands on [³H] dopamine release from rat striatal slices

| | | Percentage increase in | |
|---|---|---|---|
| | Pharmacological | [³H]dopamine release | |
| Compound | classification | 10⁻⁷M | 10⁻⁵M |
| Sibutramine | Competitive reuptake inhibitor | NS | NS |
| BTS 54 354 | Competitive reuptake inhibitor | NS | NS |
| BTS 54 505 | Competitive reuptake inhibitor | NS | NS |
| Bupropion | Competitive reuptake inhibitor | NS | NS |
| d-Amphetamine | Competitive reuptake substrate | 56 ± 9** | 138 ± 15*** |
| | releasing agent | | |
| Methamphetamine | Competitive reuptake substrate | 37 ± 10* | 140 ± 10*** |
| | releasing agent | | |
| Cocaine | Cocaine binding site inverse agonist | NS | 54 ± 19* |
| Methylphenidate | Cocaine binding site inverse agonist | NS | 24 ± 7*** |

Mean ± SEM (n ≧ 4)
*P < 0.056,
**P < 0.01,
***P < 0.001 (Williams test).
NS = Not significantly different from basal [³H]dopamine overflow.
Data taken from Heal et al (1996).

TABLE 2
Classification of various types of DAT ligand

| | | | Firing dependent | | |
|---|---|---|---|---|---|
| | | Pharmacodynamics of | effect on | Site of | Euphoriant/ |
| Classification | Examples | effect on synaptic [DA] | synaptic [DA] | action | psychostimulant |
| Competitive | Dopamine | NA | NA | Intraneuronal | NA |
| substrates | | | | via DAT | |
| Competitive | Amphetamine | Rapid, very large | No | Intraneuronal | Yes |
| substrate, | Methamphetamine | increases | | via DAT | |
| releasing agents | MDA | | | | |
| | MDMA | | | | |
| Competitive | Sibutramine | Gradual, moderate, | Yes | Extraneuronal | No |
| inhibitors | Bupropion | increases | | | |
| Inverse agonists | Cocaine | Rapid, very large | Yes | Extraneuronal | Yes |
| | Methylphenidate | increases | | | |

NA = Not applicable
[DA] = Dopamine concentration

TABLE 3
A comparison of the relative potency of cocaine as a reuptake inhibitor
of radiolabelled dopamine, noradrenaline and serotonin (5-HT)

| | [³H]Monoamine reuptake | | |
|---|---|---|---|
| | inhibition into rat brain | | |
| | synaptosomes (Ki = nM) | | |
| | Dopamine | Noradrenaline | 5-HT |
| Cocaine | 270¹ | 155¹ | 180¹ |
| | 310² | 220² | 260² |

¹Richelson & Pfenning (1984);
²Hyttel (1982)

TABLE 4
Characteristics of various DAT cocaine binding site ligands using an
intracerebral microdialysis or voltammetry functional screen

| Pharmacological | | TTX and | Max. efficacy | Reversal of |
|---|---|---|---|---|
| classification | Effect on [DA] | Ca²⁺ sensitive | versus cocaine | cocaine's effect |
| Inverse agonist | Rapid, | Yes | 100% | NA |
| (cocaine) | large increase | | | |
| Partial inverse | Gradual, moderate | Yes | <100% | Yes |
| agonist | increase | | | |
| Antagonist | No effect | — | 0% | Yes |
| Partial agonist | Gradual, | Yes | <0% | Yes |
| | moderate decrease | | | |
| Agonist | Rapid, | Yes | −100% | Yes |
| | large decrease | | | |

NA = Not applicable
TTX = Tetrodotoxin

TABLE 5
Characteristics of various DAT cocaine binding site ligands in an electrically-
stimulated, fast cyclic voltammetry screen in vivo

| Pharmacological | Effect on DA | Effect on DA | Max. efficacy | Reversal of |
|---|---|---|---|---|
| classification | release rate | reuptake rate | versus cocaine | cocaine's effect |
| Inverse agonist | Increased, | None | 100% | NA |
| (cocaine) | larger peak efflux | | | |
| Partial inverse | Moderately increased, | None | <100% | Yes |
| agonist | larger peak efflux | | | |
| Antagonist | None | None | 0% | Yes |
| Partial agonist | Moderately decreased, | Moderately | <0% | Yes |
| | lower peak efflux | increased | | |
| Full agonist | Markedly decreased, | Markedly | −100% | Yes |
| | lower peak efflux | increased | | |

NA = Not applicable
DA = Dopamine

1. A method for identifying a candidate compound for treating a disorder or condition associated with dysfunction of monoamine neurotransmission in the central nervous system, the method comprising the following steps:
a) providing a compound to be tested;
b) testing the ability of the compound to bind to the cocaine-binding site of a monoamine reuptake transporter; and
c) testing the ability of the compound to modulate the inward or outward transport of monoamine neurotransmitters via the monoamine reuptake transporter;
wherein the test compound is identified as a candidate compound for treating a disorder or condition associated with dysfunction of monoamine neurotransmission if it is able to bind to the cocaine-binding site of the monoamine reuptake transporter and modulate its activity.

2. The method according to claim 1 wherein step (c) comprises testing the ability of the compound to modulate the activity of the cocaine binding site of the monoamine reuptake transporter.

3. The method according to claim 1 or 2 wherein the monoamine is selected from the group consisting of dopamine, noradrenaline, and serotonin (5-HT).

4. The method according to claim 3 wherein the monoamine is dopamine.

5. The method according to claim 4 wherein the disorder or condition is associated with a deficit of dopamine neurotransmission in the central nervous system.

6. The method according to claim 5 wherein the disorder or condition is selected from the group comprising or consisting of Parkinson's disease, narcolepsy, attention deficit hyperactivity disorder (ADHD), borderline personality disorder, intermittent explosive disorder, antisocial personality disorder, substance abuse, kleptomania and pyromania.

7. The method according to claim 4 wherein the disorder or condition is associated with an excess of dopamine neurotransmission in the central nervous system.

8. The method according to claim 7 wherein the disorder or condition is selected from the group consisting of schizophrenia, schizo-affective disorder, schizophreniform disorder, substance abuse-induced psychotic disorder, delusional disorder, mania and shared psychotic disorder.

9. The method according to claim 3 wherein the monoamine is noradrenaline.

10. The method according to claim 9 wherein the disorder or condition is associated with a deficit of noradrenaline neurotransmission in the central nervous system.

11. The method according to claim 10 wherein the disorder or condition is selected from the group comprising disorders of impulsiveness, attention and aggression, for example attention deficit hyperactivity disorder (ADHD), borderline personality disorder, intermittent explosive disorder, antisocial personality disorder, substance abuse, kleptomania, pyromania and depression.

12. The method according to claim 9 wherein the disorder or condition is associated with an excess of noradrenaline neurotransmission in the central nervous system.

13. The method according to claim 12 wherein the disorder or condition is selected from the group comprising panic attacks, post-traumatic stress disorder, anxiety, phobias and obsessive-compulsive disorder.

14. The method according to claim 3 wherein the monoamine is serotonin (5-HT).

15. The method according to claim 14 wherein the disorder or condition is associated with a deficit of serotonin neurotransmission in the central nervous system.

16. The method according to claim 15 wherein the disorder or condition is selected from the group comprising disorders of impulsiveness, attention and/or aggression, for example borderline personality disorder, intermittent explosive disorder, antisocial personality disorder, substance abuse, kleptomania, pyromania, eating disorders (binge eating, bulimia, anorexia), anxiety, phobias, obsessive-compulsive disorder and depression.

17. The method according to claim 14 wherein the disorder or condition is associated with an excess of serotonin neurotransmission in the central nervous system.

18. The method according to claim 17 wherein the disorder or condition is migraine.

19. The method according to any one of the preceding claims wherein steps (b) and/or (c) are performed by a method selected from the group consisting of in vitro receptor binding, in vitro neurotransmitter release and/or reuptake (for example using brain slices or synaptosomes), in vitro electrophysiology, in vitro or in vivo biosensors, in vivo microdialysis or in vivo voltammetry.

20. The method according to any one of the preceding claims wherein step (c) comprises testing the ability of the compound to modulate passively the activity of the monoamine reuptake transporter.

21. The method according to any one of the preceding claims wherein step (c) comprises testing the ability of the compound to modulate actively the activity of the monoamine reuptake transporter.

22. The method according to any one of the preceding claims wherein step (c) comprises testing the ability of the compound to act as an antagonist of the monoamine reuptake transporter.

23. The method according to any one of the preceding claims wherein step (c) comprises testing the ability of the compound to act as an inverse agonist of the monoamine reuptake transporter.

24. The method according to claim 23 wherein step (c) comprises testing the ability of the compound to act as a full inverse agonist of the monoamine reuptake transporter.

25. The method according to claim 23 wherein step (c) comprises testing the ability of the compound to act as a partial inverse agonist of the monoamine reuptake transporter.

26. The method according to any one of the preceding claims wherein step (c) comprises testing the ability of the compound to act as an agonist of the monoamine reuptake transporter.

27. The method according to claim 26 wherein step (c) comprises testing the ability of the compound to act as a full agonist of the monoamine reuptake transporter.

28. The method according to claim 26 wherein step (c) comprises testing the ability of the compound to act as a partial agonist of the monoamine reuptake transporter.

29. The method according to any one of the preceding claims wherein step (c) comprises testing the ability of the compound to antagonise the effect of agonists or inverse agonists of the monoamine reuptake transporter.

30. The method according to any one of the preceding claims wherein step (c) comprises or consists of testing the ability of the compound to modulate the activity of the monoamine reuptake transporter in vitro.

31. The method according to any one of the preceding claims wherein step (c) comprises or consists of testing the ability of the compound to modulate the activity of the monoamine reuptake transporter in vivo.

32. A method according to any one of claims 20 to 31 wherein step (c) comprises or consists of testing the ability of the compound to act at the cocaine binding site on the monoamine reuptake transporter.

33. A method according to claim 32 wherein step (c) comprises or consists of testing the ability of the compound to act as an inverse agonist (full or partial), agonist (full or partial) or antagonist at the cocaine binding site on the dopamine reuptake transporter.

34. The method according to any one of the preceding claims wherein step (c) comprises testing the ability of the compound to modulate the activity of the monoamine reuptake transporter using one or more of the following techniques:
(A) In vitro measurement of spontaneous monoamine release from tissue slices, cells (or a subcellular fraction thereof) containing monoamine reuptake transporter sites by superfusion;
(B) In vitro measurement of monoamine reuptake from tissue slices, cells (or a subcellular fraction thereof) containing monoamine reuptake transporter sites by superfusion;
(C) In vitro measurement of electrically-evoked release of monoamine from tissue slices, cells (or a subcellular fraction thereof) containing monoamine reuptake transporter sites by superfusion;
(D) In vitro measurement of spontaneous and/or electrically-evoked monoamine efflux from tissue slices, cells (or a subcellular fraction thereof) containing monoamine reuptake transporter sites by electrophysiological techniques;
(E) In vitro and/or in vivo measurement of spontaneous and/or electrically evoked monoamine efflux using one or more biosensors;
(F) In vivo measurement of cell-firing dependent and cell-firing independent monoamine efflux by microdialysis in animals;
(G) in vivo measurement of spontaneous and/or electrically-evoked monoamine efflux in animals by voltammetric techniques.

35. The method according to claim 34 wherein (A), (B) and (C) comprise the in vitro measurement of release or reuptake of a labelled monoamine.

36. The method according to claim 34 wherein the biosensors of (E) are coated with enzymes, antibodies and/or neurotransmitter receptors.

37. The method according to any one of claims 34 to 36 wherein step (c) comprises one or more of the following technique options/combinations:
A Alone, B alone, C alone, D alone, E alone, F alone, G alone, A+B, A+C, A+D, A+B+C, A+B+C+D, A+B+D, B+C, B+D, C+B, C+D, A+C+E (in vitro), A+C+E (in vivo), A+C+F, A+C+G, A+B+C+E (in vitro), A+B+C+E (in vivo), A+B+C+F, A+B+C÷G, A+D+E (in vitro), A+D+E (in vivo), A+D+F, A+D+G, A+B+D+E (in vitro), A+B+D+E (in vivo), A+B+D+F, A+B+D+G

38. The method according to any one of claims 34 to 37 wherein the cells containing monoamine reuptake transporter sites are in or derived from tissue slices.

39. The method according to claim 38 where the tissue slices are from the brain, for example from dopaminergic regions of the brain.

40. The method according to any one of claims 34 to 39 wherein the cells containing monoamine reuptake transporter sites are maintained in culture.

41. The method according to any one of claims 34 to 40 wherein the cells are selected from the group consisting of primary cells and immortalised cells (i.e. cell lines).

42. The method according to any one of claims 34 to 41 wherein the cells are genetically modified to express a monoamine reuptake transporter.

43. The method according to any one of claims 34 to 42 where the cells containing monoamine reuptake transporter sites are blood cells.

44. The method according to any one of claims 34 to 42 where the cells containing monoamine reuptake transporter sites are in or derived from renal blood vessels.

45. The method according to any one of claims 34 to 40 wherein monoamine release or reuptake is measured in synaptosomes.

46. The method according to any one of claims 34 to 45 where the cells containing monoamine reuptake transporter sites are from a human.

47. The method according to any one of claims 34 to 45 where the cells containing monoamine reuptake transporter sites are from a non-human species, for example a rodent such as a mouse or a rat.

48. The method according to any one of claims 34 to 37 wherein the in vivo measurements are performed in the brain.

49. The method according to claim 48 wherein the in vivo measurements are performed in regions of the brain rich in cells which contain monoamine reuptake transporters

50. The method according to claim 49 wherein the in vivo measurements are performed in dopaminergic regions of the brain, e.g. the basal ganglia.

51. The method according to any one of claims 48 to 50 wherein the in vivo measurements are performed in a human or in a non-human species for example, a rodent such as a mouse or a rat.

52. The method according to any one the preceding claims wherein step (c) comprises testing the ability of the test compound at different doses to modulate the activity of the monoamine reuptake transporter.

53. The method according to any one of claims 34 to 47 wherein in vitro measurement of release of monoamine from tissue by superfusion is performed using a high dose of the test compound (e.g. 1×10⁻⁵ M) and a low dose of the test compound (e.g. 1×10⁻⁷ M).

54. The method according to any one of the preceding claims further comprising counter-screening the test compounds for adverse or undesirable properties.

55. The method according to any one of the preceding claims further comprising step (d) of formulating a compound identified as a candidate compound for treating a disorder or condition associated with dysfunction of monoamine neurotransmission in the central nervous system into a pharmaceutical composition.

56. A compound identified by a method according to any one of the preceding claims.

57. A compound according to claim 56 wherein the compound is a full or partial inverse agonist of the cocaine binding site of a monoamine reuptake transporter.

58. A compound according to claim 56 wherein the compound is a full or partial agonist of the cocaine binding site of a monoamine reuptake transporter.

59. A compound according to claim 56 wherein the compound is an antagonist of ligands which act as agonists or inverse agonists of the cocaine binding site of a monoamine reuptake transporter.

60. A compound according to claim 59 wherein the ligand which acts as an inverse agonist of the cocaine binding site is cocaine or a related compound (e.g. methylphenidate).

61. A pharmaceutical composition comprising a compound according to any one of claims 56 to 60 and a pharmaceutically-acceptable carrier or excipient.

62. A compound according to any one of claims 56 to 60 for use in medicine.

63. Use of compound according to any one of claims 56 to 60 in the manufacture of a medicament for treating a disorder or condition associated with dysfunction of monoamine neurotransmission in the central nervous system.

64. The use according to claim 63 wherein the monoamine is selected from the group consisting of the dopamine, noradrenaline and serotonin.

65. The use according to claim 64 wherein the monoamine is dopamine.

66. The use according to claim 65 wherein the disorder or condition is associated with a deficit of dopamine neurotransmission in the central nervous system.

67. The use according to claim 66 wherein the disorder or condition is selected from the group comprising Parkinson's disease, narcolepsy, attention deficit hyperactivity disorder (ADHD), borderline personality disorder, intermittent explosive disorder, antisocial personality disorder, substance abuse, kleptomania and pyromania.

68. The use according to claim 65 wherein the disorder or condition is associated with an excess of dopamine neurotransmission in the central nervous system.

69. The use according to claim 64 wherein the disorder or condition is selected from the group comprising schizophrenia, schizo-affective disorder, schizophreniform disorder, substance abuse-induced psychotic disorder, delusional disorder, mania and shared psychotic disorder.

70. The use according to claim 64 wherein the monoamine is noradrenaline.

71. The use according to claim 70 wherein the disorder or condition is associated with a deficit of noradrenaline neurotransmission in the central nervous system.

72. The use according to claim 71 wherein the disorder or condition is selected from the group comprising disorders of impulsiveness, attention and aggression, for example attention deficit hyperactivity disorder (ADHD), borderline personality disorder, intermittent explosive disorder, antisocial personality disorder, substance abuse, kleptomania, pyromania and depression.

73. The use according to claim 70 wherein the disorder or condition is associated with an excess of noradrenaline neurotransmission in the central nervous system.

74. The use according to claim 73 wherein the disorder or condition is selected from the group comprising panic attacks, post-traumatic stress disorder, anxiety, phobias and obsessive-compulsive disorder.

75. The use according to claim 64 wherein the monoamine is serotonin (5-HT).

76. The use according to claim 75 wherein the disorder or condition is associated with a deficit of serotonin neurotransmission in the central nervous system.

77. The use according to claim 76 wherein the disorder or condition is selected from the group comprising disorders of impulsiveness, attention and/or aggression, for example borderline personality disorder, intermittent explosive disorder, antisocial personality disorder, substance abuse, kleptomania, pyromania, eating disorders (binge eating, bulimia, anorexia), anxiety, phobias, obsessive-compulsive disorder and depression.

78. The use according to claim 75 wherein the disorder or condition is associated with an excess of serotonin neurotransmission in the central nervous system.

79. The use according to claim 78 wherein the disorder or condition is migraine.

80. A method or use substantially as described herein with reference to the description and examples.

81. A compound or pharmaceutical composition substantially as described herein with reference to the description and examples.
